(19) United States
(12) Patent Application Publication
(10) Pub. No.: US 2014/0066778 A1
(43) Pub. Date: Mar. 6, 2014

(54) ULTRASONIC TRANSDUCER DEVICE, PROBE, ELECTRONIC INSTRUMENT, AND ULTRASONIC DIAGNOSTIC DEVICE

(71) Applicant: SEIKO EPSON CORPORATION, TOKYO (JP)

(72) Inventor: Tsutomu NISHIWAKI, Azumino (JP)

(73) Assignee: SEIKO EPSON CORPORATION, TOKYO (JP)

(21) Appl. No.: 14/010,755

(22) Filed: Aug. 27, 2013

(30) Foreign Application Priority Data
Aug. 28, 2012 (JP) 2012-187465

(51) Int. Cl. A61B 8/00

(52) U.S. Cl. CPC A61B 8/4483
USPC 600/459

(57) ABSTRACT

An ultrasonic transducer device includes a substrate, a vibrating film, a piezoelectric element, an input section and a detection section. The substrate has a plurality of openings. The vibrating film provided in each of the openings to cover a corresponding one of the openings. The piezoelectric element is provided in each of the openings on the vibrating film. The input section is configured and arranged to input a drive signal to a part of piezoelectric elements among the piezoelectric elements. The detection section is configured and arranged to detect vibration of the piezoelectric elements, in which the drive signal is not inputted, among the piezoelectric elements while the drive signal is inputted to the part of the piezoelectric elements among the piezoelectric elements.

### CROSS-REFERENCE TO RELATED APPLICATIONS

This application claims priority to Japanese Patent Application No. 2012-187465 filed on Aug. 28, 2012. The entire disclosure of Japanese Patent Application No. 2012-187465 is hereby incorporated herein by reference.

### BACKGROUND

1. Technical Field

The present invention relates to an ultrasonic transducer device, and also relates to a probe, an electronic instrument, and an ultrasonic diagnostic device, and the like using the ultrasonic transducer device.

2. Related Art

An ultrasonic transducer device can be provided with one substrate. Openings are formed in the substrate. An ultrasonic transducer element is provided in each opening. An ultrasonic transducer element is provided with a vibrating film. The vibrating film covers the openings from the surface of the substrate. A piezoelectric element is provided on the vibrating film. The vibration of the vibrating film in each ultrasonic transducer element is generated by the function of the piezoelectric element. The ultrasonic wave is generated in response to the vibration of the vibrating film. In this kind of the ultrasonic transducer device, a piezoelectric film of the piezoelectric element can be thinly formed.

### SUMMARY

As described in Japanese Laid-open Patent Application Publication No. 2009-302445, a polarization is established in the piezoelectric film. When the piezoelectric film is thinly formed, the coercive voltage is relatively low so that an amount of the polarization tends to be reduced due to disturbance such as electromagnetic noise or temperature, and the like. It is concerned that the amount of the polarization is getting to be reduced over time. The reduction of the polarization becomes the deterioration of the sensitivity so that the deterioration of the sensitivity leads to get worse on the measurement accuracy. However, the sensitivity of the piezoelectric element could not be detected without using a device, which is different from the ultrasonic transducer device, for example, a calibrating apparatus.

According to at least one aspect of the present invention, a sensitivity of a piezoelectric element can be detected without using a device, which is different from an ultrasonic transducer device.

(1) One aspect of the present invention relates to an ultrasonic transducer device comprising: a substrate having a plurality of openings; a vibrating film covering the openings; a piezoelectric element being provided in each opening on the vibrating film; an input section inputting a drive signal to a part of piezoelectric elements among the piezoelectric elements; and a detection section detecting vibration of the piezoelectric elements, in which the drive signal is not inputted, among the piezoelectric elements while the drive signal is inputted to the part of the piezoelectric elements among the piezoelectric elements.

A part of the piezoelectric elements is deformed in response to the supply of the drive signal. The deformation of the piezoelectric element leads to the deformation of the corresponded vibrating film. The substrate is deformed in response to the deformation of the vibrating film. The deformation of the substrate leads to the deformation of another vibrating film. The deformation of another vibrating film generates stress in a piezoelectric element, in which the drive signal is not inputted. An electromotive voltage is generated in the piezoelectric element, in which the drive signal is not inputted. By detecting the electromotive voltage, the sensitivity of the piezoelectric element can be detected without using a device, which is different from the ultrasonic transducer device, for example, a calibrating apparatus.

(2) The ultrasonic transducer device can be provided with a control processing section. The control processing section can determine the sensitivity of the piezoelectric elements based on the detection result that the vibration of a piezoelectric element in which the drive signal is not inputted is detected. Because of this, the quality of the sensitivity can be determined.

(3) In the ultrasonic transducer device, the plurality of openings is arranged in a matrix pattern or a line pattern in plan view of a thickness direction of the substrate. The detection section can detect vibration of a piezoelectric element that is located adjacent to the part of piezoelectric elements, in which the drive signal is inputted, among the piezoelectric elements, in which the drive signal is not inputted. The part of the piezoelectric elements, in which the drive signal is inputted, leads to the deformation of the piezoelectric elements, in which the drive signal is not inputted, securely.

(4) The detection section can detect vibration of a piezoelectric element that is located between two piezoelectric elements, in which the drive signal is inputted, among the piezoelectric elements, in which the drive signal is not inputted, and is also located adjacent to the two piezoelectric elements. Because of this, the deformation force is applied to the piezoelectric element, in which the drive signal is not inputted, from the both sides. Therefore, the stress of the piezoelectric element can be increased by the supply of the drive signal at once. The electromotive voltage of the piezoelectric element is increased. As a result, the detection accuracy of the sensitivity can be increased.

(5) When the control processing section determines that the sensitivity of a piezoelectric element, in which the drive signal is not inputted, is less than a predetermined value, a voltage for polarization can be supplied to the piezoelectric element, in which the drive signal is not inputted. A polarization is established in the piezoelectric element prior to use. The amount of the polarization is reduced over time. As a result, the sensitivity of the piezoelectric element is reduced. Therefore, in a case that the sensitivity of the piezoelectric element is reduced less than the predetermined value, when the voltage for polarization is supplied to the piezoelectric element, a sufficient polarization can be established again in the piezoelectric element. The good sensitivity of the piezoelectric element can be maintained.

(6) When the control processing section determines that the sensitivity of a piezoelectric element, in which the drive signal is not inputted, is less than the predetermined value, a notification signal indicating that the sensitivity is less than the predetermined value can be outputted. The sensitivity deterioration of the piezoelectric element can be notified to outside from the control processing section. A user can recognize the sensitivity deterioration of the piezoelectric element based on the notification.

(7) The ultrasonic transducer device can be provided with the control processing section. The control processing section can output a notification signal based on the detection result that the vibration of a piezoelectric element, in which the drive signal is not inputted, was detected. Because of this, the detection result can be notified to the outside from the control processing section. The user can determine the sensitivity deterioration of the piezoelectric element based on this notification.

(8) Another aspect of the present invention relates to an ultrasonic transducer device comprising: a substrate having a first opening, a second opening, and a partition part, which is wedged between the first opening and the second opening; a vibrating film covering the first opening and the second opening; a first piezoelectric element being provided on the vibrating film including a position overlapped with the first opening in plan view as viewed along the thickness direction of the substrate; a second piezoelectric element being provided on the vibrating film including a position overlapped with the second opening in plan view as viewed along the thickness direction of the substrate; an input section inputting a drive signal to the first piezoelectric element; and a detection section detecting vibration of the second piezoelectric element, in which the drive signal is not inputted, while the drive signal is inputted to the first piezoelectric element. The partition part has a shape that the thickness of the thickness direction of the substrate is larger than a minimum value of distance between the first opening and the second opening in plan view as viewed along the thickness direction of the substrate.

The first piezoelectric element is deformed in response to the supply of the drive signal. The deformation of the first piezoelectric element leads to the deformation of the vibrating film overlapped with the first opening. The partition part is deformed in response to the vibrating film. The deformation of the partition part leads to the deformation of the vibrating film overlapped with the second opening. The deformation of the vibrating film overlapped with the second opening generates the stress in the second piezoelectric element. The electromotive voltage is generated in the second piezoelectric element, in which the drive signal is not inputted. By detecting the electromotive voltage, the sensitivity of the second piezoelectric element can be detected without using a device, which is different from the ultrasonic transducer device, for example, calibrating apparatus.

(9) Further, another aspect of the present invention relates to an ultrasonic transducer device comprising: a substrate having a plurality of first openings, which is arranged in a matrix pattern or a line pattern, and a second opening, which is arranged in an outside of an outline of an area where the plurality of first openings are arranged; a vibrating film covering the first openings and the second opening; a first piezoelectric element being provided on the vibrating film in each of the plurality of first openings in plan view as viewed along the thickness direction of the substrate; a second piezoelectric element being provided on the vibrating film including a position overlapped with the second opening in plan view as viewed along the thickness direction of the substrate; an input section inputting a drive signal to the first piezoelectric elements; and a detection section detecting vibration of the second piezoelectric element, in which the drive signal is not inputted, while the drive signal is inputted to the first piezoelectric elements.

The first piezoelectric elements are deformed in response to the supply of the drive signal. The deformation of the first piezoelectric elements leads to the deformation of the vibrating film overlapped with the first openings. The substrate is deformed in response to the deformation of the vibrating film. The deformation of the substrate leads to the deformation of the vibrating film overlapped with the second opening. The deformation of the vibrating film overlapped with the second opening generates stress in the second piezoelectric element. The electromotive voltage is generated in the second piezoelectric element, in which the drive signal is not generated. By detecting the electromotive voltage, the sensitivity of the second piezoelectric element can be detected without using a device, which is different from the ultrasonic transducer device, for example, a calibrating apparatus.

(10) In the ultrasonic transducer device, the first opening and the second opening can be formed in the same shape, and the first piezoelectric element and the second piezoelectric element can be formed in the same structure. Because of this, the characteristics of the second piezoelectric element can be correlated with the characteristics of the first piezoelectric. The characteristics of the second piezoelectric element can be influenced from the characteristics of the first piezoelectric element with high accuracy.

(11) Further, another aspect of the present invention relates to an ultrasonic transducer device comprising: a substrate having a plurality of first openings, which is arranged in a matrix pattern or a line pattern, a second opening, which is arranged in an outside of an outline of a region where the plurality of first openings is arranged, and a third opening, which is arranged in the outside of the outline of the region where the plurality of first openings is arranged, and is also arranged in a position closer to the second opening than the first openings; a vibrating film covering the first openings, the second opening, and third opening; a first piezoelectric element being provided on the vibrating film in each of the plurality of first openings in plan view of a thickness direction of the substrate; a second piezoelectric element being provided on the vibrating film including a position overlapped with the second opening in plan view as viewed along the thickness direction of the substrate; a third piezoelectric element being provided on the vibrating film including a position overlapped with the third opening in plan view as viewed along the thickness direction of the substrate; an input section inputting a drive signal to the third piezoelectric element; and a detection section detecting vibration of the second piezoelectric element, in which the drive signal is not inputted, while the drive signal is inputted to the third piezoelectric element.

The third piezoelectric element is deformed in response to the supply of the drive signal. The deformation of the third piezoelectric element leads to the deformation of the vibrating film of the third opening. The substrate is deformed in response to the deformation of the vibrating film. The deformation of the substrate leads to the deformation of the vibrating film of the second opening. The deformation of the vibrating film of the second opening generates stress in the second piezoelectric element. The electromotive voltage is generated in the second piezoelectric element, in which the drive signal is not inputted. By detecting the electromotive voltage, the sensitivity of the second piezoelectric element can be detected without using a device, which is different from the ultrasonic transducer device, for example, a calibrating apparatus. Generally, the characteristics of the second piezoelectric element are reflected from the characteristics of the first piezoelectric element so that the sensitivity of the first piezoelectric element can be presumed based on the sensitivity of the second piezoelectric element.

(12) The third piezoelectric element can have larger area than the second piezoelectric element in plan view as viewed along the thickness direction of the substrate. Because of this, a greater deformation force can be applied to the second piezoelectric element. As a result, the accuracy of detection for vibration can be improved.

(13) The ultrasonic transducer device can be provided with the control processing section. The control processing section can determine the sensitivity of the first piezoelectric element based on the detection result that the vibration of the second piezoelectric element in which the drive signal is not inputted is detected. Because of this, the quality of the sensitivity can be determined.

(14) When the control processing section determines that the sensitivity of the first piezoelectric element is less than a predetermined value, a voltage for polarization can be supplied to the first piezoelectric element. A polarization is established in the piezoelectric element prior to use. The amount of the polarization is reduced over time. As a result, the sensitivity of the piezoelectric element is reduced. Therefore, in a case that the sensitivity of the piezoelectric element is reduced less than the predetermined value, when the voltage for polarization is supplied to the piezoelectric element, a sufficient polarization can be established again in the piezoelectric element. The good sensitivity of the piezoelectric element can be maintained.

(15) When the control processing section determines that the sensitivity of the first piezoelectric element is less than the predetermined value, a notification signal indicating that the sensitivity is less than the predetermined value can be outputted. The sensitivity deterioration of the piezoelectric element can be notified to outside from the control processing section. A user can recognize the sensitivity deterioration of the piezoelectric element based on the notification.

(16) Any of the ultrasonic transducer devices can be used by being incorporated in a probe. The probe can be provided with an ultrasonic transducer device and a case that supports the ultrasonic transducer device.

(17) The ultrasonic transducer device can be used by being incorporated in an electronic instrument. The electronic instrument can be provided with the ultrasonic transducer device and a processor that is connected to the ultrasonic transducer device and processes an output of the ultrasonic transducer device.

(18) The ultrasonic transducer device can be used by being incorporated in the ultrasonic diagnostic device. The ultrasonic diagnostic device can be provided with the ultrasonic transducer device, a processor that is connected to the ultrasonic transducer device, processes an output of the ultrasonic transducer device, and generates an image, and a display device that displays the image.

(19) Any of the ultrasonic transducer devices can be used by being incorporated in a probe head. The probe head can be provided with the ultrasonic transducer device, and a case that supports the ultrasonic transducer device.

(20) Further, another aspect of the present invention relates to an ultrasonic transducer device comprising: a substrate having a plurality of openings partitioned by a partition part; a vibrating film covering the openings; a piezoelectric element being provided in each opening on the vibrating film; an input section inputting a drive signal to a part of piezoelectric elements among a plurality of piezoelectric elements; and a detection section detecting vibration of the piezoelectric element, in which the drive signal is not inputted, while the drive signal is inputted in the part of piezoelectric elements. The vibration of the vibrating film vibrated by inputting the drive signal to the part of piezoelectric elements deforms the partition part so as to vibrate the piezoelectric elements, in which the drive signal is not inputted.

The part of the piezoelectric elements is deformed in response to the supply of the drive signal. The deformation of the piezoelectric elements leads to the vibration of the corresponded vibrating film. The partition part is deformed in response to the vibration of the vibrating film. The deformation of the partition part leads to the deformation of another vibrating film. The deformation of another vibrating film generates stress in the piezoelectric elements, in which the drive signal is not inputted. The electromotive voltage is generated in the piezoelectric elements, in which the drive signal is not inputted. By detecting the electromotive voltage, the sensitivity of the piezoelectric elements can be detected without using a device, which is different from the ultrasonic transducer device, for example, calibrating apparatus.

### BRIEF DESCRIPTION OF THE DRAWINGS

Referring now to the attached drawings which form a part of this original disclosure:

FIG. 1 is an appearance diagram schematically showing a concrete example of an electronic instrument, that is, an ultrasonic diagnostic device according to one embodiment.

FIG. 2 is an enlarged front view of an ultrasonic probe.

FIG. 3 is an enlarged plane view of an ultrasonic transducer element unit according to the first embodiment.

FIG. 4 is a cross-sectional diagram along an A-A line of FIG. 3.

FIG. 5 is a block diagram schematically showing a circuit configuration of an ultrasonic diagnostic device.

FIG. 6 is a flowchart schematically showing operations of a sensitivity detection mode.

FIG. 7 is an enlarged cross-sectional diagram schematically showing a structure of the sensitivity detection mode corresponding to FIG. 4.

FIG. 8 is a partial enlarged vertical sectional diagram schematically showing a flexible film and the second electric conductor formed on a silicon wafer.

FIG. 9 is a partial enlarged vertical sectional diagram schematically showing a piezoelectric element and the first electric conductor formed on the silicon wafer.

FIG. 10 is a partial enlarged vertical sectional diagram schematically showing a film of an electrical conducing material covering the silicon wafer.

FIG. 11 is a partial enlarged vertical sectional diagram schematically showing openings and a reinforcing plate formed in the silicon wafer.

FIG. 12 is an enlarged plane view of an ultrasonic transducer element unit according to the second embodiment.

FIG. 13 is a cross-sectional diagram along a B-B line of FIG. 12.

FIG. 14 is a block diagram schematically showing a circuit configuration of an ultrasonic diagnostic device in which the ultrasonic transducer element unit according to the second embodiment is used.

FIG. 15 is an enlarged plane view of an ultrasonic transducer element unit according to the third embodiment.

FIG. 16 is a block diagram schematically showing a circuit configuration of an ultrasonic diagnostic device according to another embodiment.

### DETAILED DESCRIPTION OF EXEMPLARY EMBODIMENTS

Hereinafter, one embodiment of the present invention will be described with reference to the attached drawings. The present embodiment described below shall not be construed as unreasonably limiting the subject matter of the present invention described in the claims, and all the elements described in the present embodiment are not necessarily essential to the solving means of the present invention.

### (1) Overall Configuration of Ultrasonic Diagnostic Device

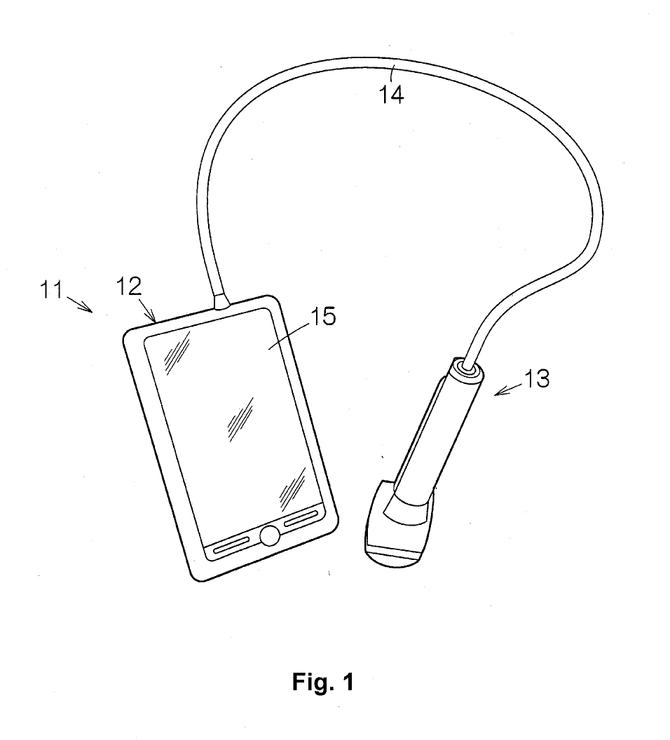

FIG. 1 schematically shows a concrete example of an electronic instrument, that is, an ultrasonic diagnostic device 11 according to one embodiment. The ultrasonic diagnostic device 11 is provided with a device terminal 12 and an ultrasonic probe (probe) 13. The device terminal 12 and the ultrasonic probe 13 are reciprocally connected by a cable 14. The device terminal 12 and the ultrasonic probe 13 are communicated by an electric signal through the cable 14. The device terminal 12 is provided with a display panel (display device) 15. A screen of the display panel 15 is exposed in the surface of the device terminal 12. As described later, in the device terminal 12, an image is generated based on an ultrasonic wave detected in the ultrasonic probe 13. An imaging of the detection result is displayed in the screen of the display panel 15.

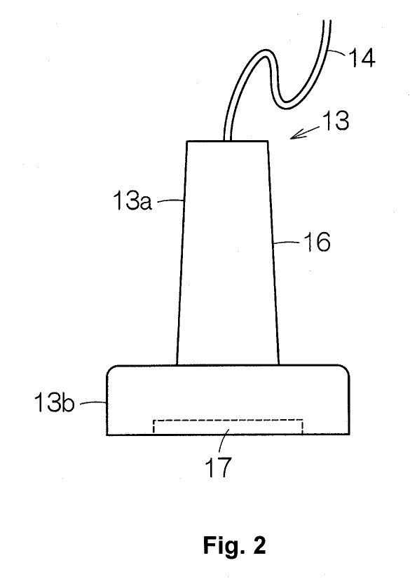

As shown in FIG. 2, the ultrasonic probe 13 has a case 16. In the case 16, an ultrasonic transducer element unit (hereinafter referred to as “element unit”) 17 is stored. The surface of the element unit 17 can be exposed in the surface of the case 16. The element unit 17 outputs an ultrasonic wave from the surface and receives a reflection wave of the ultrasonic wave. Also, the ultrasonic probe 13 can be provided with a probe head 13b that is detachably connected to a probe main body 13a. At this point, the element unit 17 can be incorporated in the case 16 of the probe head 13b.

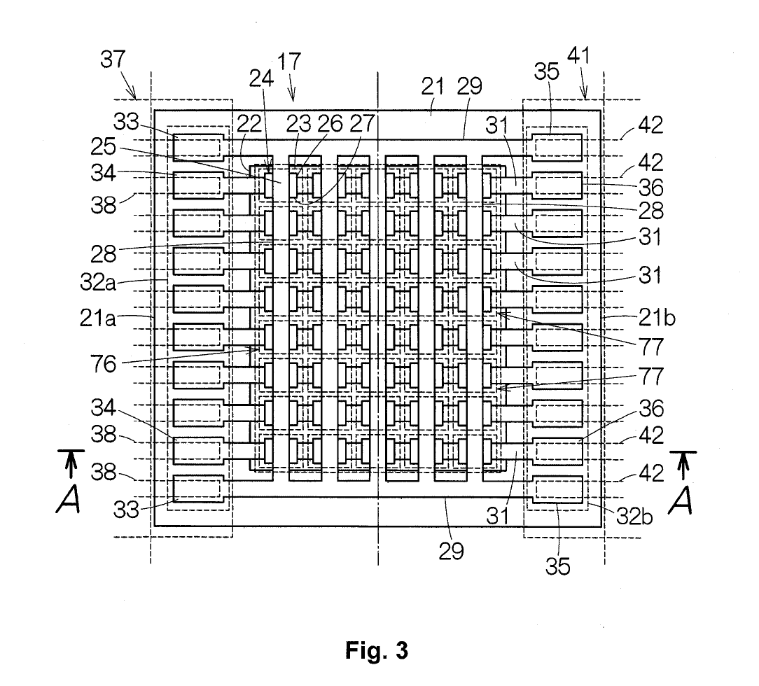

FIG. 3 schematically shows a plane view of the element unit 17 according to the first embodiment. The element unit 17 is provided with a substrate base 21. An element array 22 is formed in the substrate base 21. The element array 22 is configured in an arrangement of the ultrasonic transducer elements (hereinafter, referred to as “element”) 23. The arrangement is formed by matrix of a plural number of rows and a plural number of columns. Each element 23 is provided with a piezoelectric element section 24. The piezoelectric element section 24 is provided with an upper electrode 25, a lower electrode 26, and a piezoelectric film 27. The piezoelectric film 27 is wedged between the upper electrode 25 and the lower electrode 26 in each element 23. The element unit 17 is configured by one ultrasonic transducer element chip.

A plural number of the first electric conductors 28 are formed on the surface of the substrate base 21. The first electric conductors are reciprocally extended parallel in a direction of rows in the arrangement. In each element 23 of one row, one first electric conductor 28 is allocated. The one first electric conductor 28 is mutually connected to the piezoelectric film 27 of the element 23 parallel in the direction of rows in the arrangement. The first electric conductor 28 forms an upper electrode in each element 23. The both end parts of the first electric conductor 28 are respectively connected to a pair of extraction wirings 29. The extraction wirings 29 are reciprocally extended in parallel in the direction of columns in the arrangement. Therefore, all of the first electric conductors 28 have the same length. Because of this, the upper electrode 25 is mutually connected to all of the elements 23 in the matrix.

A plural number of the second electric conductors 31 are formed on the surface of the substrate base 21. The second electric conductors 31 are reciprocally extended in parallel to the direction of columns in the arrangement. One second electric conductor 31 is allocated in each one line of the element 23. One second electric conductor 31 is mutually provided in the piezoelectric film 27 of the element 23 in parallel to the direction of columns in the arrangement. A power distribution of the elements 23 is switched in each column. A line scan or a sector scan is realized in response to the power distribution switch. The elements 23 in one column simultaneously output an ultrasonic wave so that number in one column, that is, number of lines in the arrangement can be determined in response to an output level of the ultrasonic wave. For example, the number of lines may be set approximately 10 to 15 lines. In the drawing, it is omitted to 5 lines. The row number of the arrangement can be determined in response to a range of scanning. For example, the number of lines may be set 128 lines or 256 lines. In the drawing, it is omitted to 8 lines. Also, a zigzag pattern may be established in the arrangement. In the zigzag pattern, a group of the elements 23 in an even column may be displaced with respect to a group of the elements 23 in an odd column by one-half of the row pitch. The number of the elements in one of an odd column and an even column may be smaller than the number of the elements in the other of an odd column and an even column by one. In addition, the functions of the upper electrode 25 and the lower electrode 26 may be switched. That is, the lower electrode is mutually connected to the elements 23 of the entire matrix, and on the other hand, the upper electrode may be mutually connected to the elements 23 in each column of the arrangement.

The outline of the substrate base 21 has a first side 21a and a second side 21b that are opposed and partitioned by a pair of straight lines in parallel to each other. One line of a first terminal array 32a is provided between the first side 21a and an outline of an element array 22. One line of a second terminal array 32b is provided between the second side 23b and the outline of the element array 22. The first terminal array 32a can form one line parallel to the first side 21a. The second terminal array 32b can form one line parallel to the second side 21b. The first terminal array 32a is configured by a pair of upper electrode terminals 33 and a plurality of lower electrode terminals 34. In the same manner, the second terminal array 32b is configured by a pair of upper electrode terminals 35 and a plurality of lower electrode terminals 36. The respective upper electrode terminals 33, 35 are connected to both ends of one extraction wiring 29. It is sufficient for the extraction wiring 29 and the upper electrode terminals 33, 35 to be formed plane-symmetrically with respect to a vertical plane that bisects the element array 22. The respective lower electrode terminals 34, 36 are connected to both ends of one second electric conductor 31. It is sufficient for the second electric conductor 31 and the lower electrode terminals 34, 36 to be formed plane-symmetrically with respect to a vertical plane that bisects the element array 22. Here, the outline of the substrate base 21 is formed to be a rectangular. The outline of the substrate base 21 may be a square or trapezoid.

A first flexible printed circuit board (hereafter referred to as “the first circuit board”) 37 is connected to the substrate base 21. The first circuit board 37 is coupled with to the first terminal array 32a. Conductive lines, that is, first signal lines 38 are formed corresponding to the upper electrode terminals 33 and the lower electrode terminals 34, respectively in one end of the first circuit board 37. The first signal lines 38 are respectively opposed to the upper electrode terminals 33 and the lower electrode terminals 34, and respectively bonded thereto. Similarly, the second flexible printed circuit board (hereinafter referred to as “the second circuit board”) 41 is coupled with the substrate base 21. The second circuit board 41 is coupled with the second terminal array 32b. Conductive lines, that is, second signal lines 42 are formed at one end of the second circuit board 41 corresponding to the upper electrode terminals 35 and the lower electrode terminals 36, respectively. The second signal lines 42 are respectively opposed to the upper electrode terminals 35 and the lower electrode terminals 36, and respectively bonded thereto.

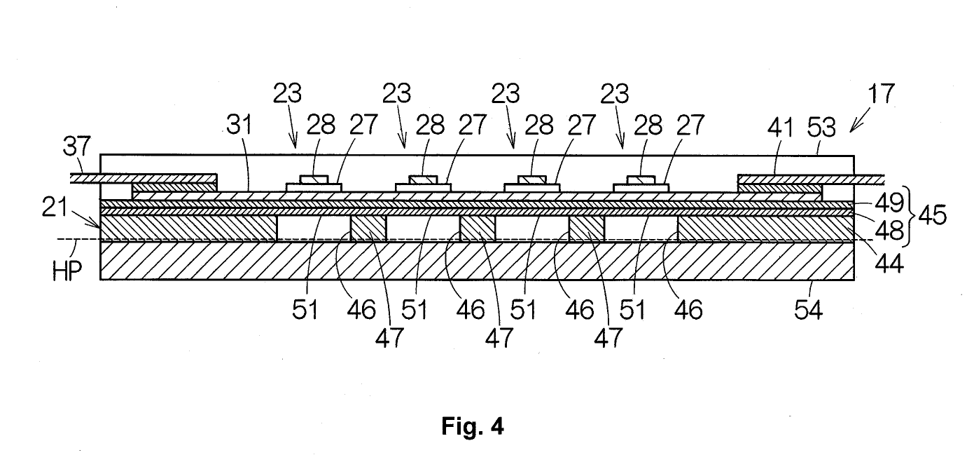

As shown in FIG. 4, the substrate base 21 is provided with a flexible film 45. The flexible film 45 is entirely formed on the surface of the substrate 44. An opening 46 is formed in each of in the elements 23 in the substrate 44. The openings 46 are arranged in an array pattern with respect to the substrate 44. An outline of an area where the openings 46 are arranged corresponds to the outline of the element array 22. A partition wall (partition part) 47 is formed between two openings 46 that are adjacent to each other. The adjacent openings 46 are divided by the partition wall 47. The thickness of the partition wall 47 corresponds to a space of the openings 46. The partition wall 47 defines two wall surfaces in the plane that reciprocally expand in parallel. The wall thickness corresponds to a distance of two wall surfaces. That is, the wall thickness can be defined by a length of vertical line that intersects the wall surfaces and is wedged between the wall surfaces.

The flexible film 45 is constructed of a silicon oxide (SiO₂) layer 48 layered on the surface of the substrate 44, and a zirconium oxide (ZrO₂) layer 49 layered on a surface of the silicon oxide layer 48. The flexible film 45 contacts the openings 46. Because of this, a part of the flexible film 45 with respect to the outline of the openings 46 forms a vibrating film 51. In the flexible film 45, the vibrating film 51 is a part that faces onto the opening 46 so that it can vibrate in the thickness direction of the substrate 44. The film thickness of the silicon oxide layer 48 can be determined based on resonance frequency.

The second electric conductor 31, the piezoelectric film 27, and the first electric conductor 28 are layered on a surface of the vibrating film 51 in this order. As for the second electric conductor 31, for example, a layered film of titanium (Ti), iridium (Ir), platinum (Pt), and titanium (Ti) can be used. The piezoelectric film 27 may be formed of, for example, piezoelectric zirconate titanate (PZT). The first electric conductor 28 can be formed of, for example, iridium (Ir). Another conductive material may be used in the first electric conductor 28 and the second electric conductor 31, and another piezoelectric material may be used in the piezoelectric film 27. Here, the piezoelectric film 27 completely covers the second electric conductor 31 under the first electric conductor. The function of the piezoelectric film 27 prevents short circuit between the first electric conductor 28 and the second electric conductor 31 from occurring.

A protective film 53 is layered on the surface of the substrate base 21. The protective film 51 covers, for example, the entire surface of the substrate base 21. As a result, the protective film 51 covers the element array 22, the first and the second terminal arrays 32a, 32b, the first and the second circuit boards 37, 41. For example, a silicone resin film may be used for the protective film 53. The protective film 53 protects the structure of the element array 22, the bonding of the first terminal array 32a and the first circuit board 37, and the bonding of the second terminal array 32b and the second circuit board 41.

A reinforcing plate 54 is fixed on the back surface of the substrate base 21. The back surface of the substrate base 21 is overlapped on the surface of the reinforcing plate 54. The reinforcing plate 54 closes the openings 46 on the back surface of the element unit 17. The reinforcing plate 54 can be provided with a rigid substrate. The reinforcing plate can be formed of, for example, silicon substrate. The plate thickness of the substrate base 21 is set, for example, approximately 100 μm, and the plate thickness of the reinforcing plate 54 is set, for example, approximately 100 to 150 μm. Here, the partition wall 47 is bonded to the reinforcing plate 54. The reinforcing plate 54 is bonded to the respective partition walls 47 in at least one bonding region. Adhesive can be used for the bonding.

### (2) Circuit Configuration of Ultrasonic Diagnostic Device

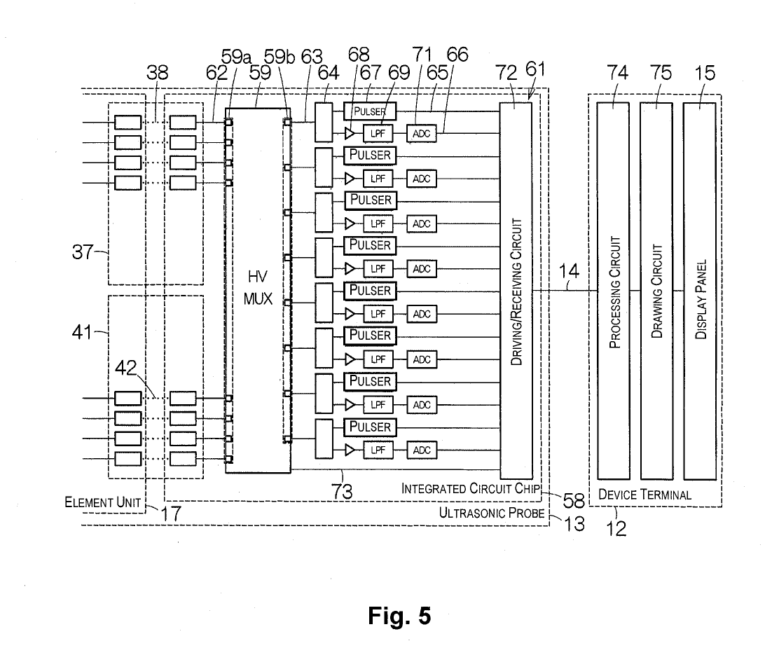

As shown in FIG. 5, the ultrasonic diagnostic device 11 is provided with an integrated circuit chip 58 that is electrically connected to the element unit 17. The integrated circuit chip 58 is provided with a multiplexer 58, and a transmitting and receiving circuit 61. The multiplexer 59 has a group of ports 59a on the element unit 17 side, and a group of ports 59b on the transmitting and receiving circuit 61 side. The first signal lines 38 and the second signal lines 42 are connected to the group of ports 59a on the element unit 17 side via the wirings 62. In this manner, the group of ports 59a is connected to the element array 22. Here, signal lines 63, which are a prescribed number in the integrated circuit board 55, are connected to the group of ports 59b on the transmitting and receiving circuit 61 side. The prescribed number corresponds to a column number of the elements 23 output at the same time as scanning is conducted. The multiplexer 59 controls the interconnection between the ports on the cable 14 side and the ports on the element unit 17 side.

The transmitting and receiving circuit 61 is provided with selecting switches 64 of a prescribed number. The selecting switches are respectively connected to corresponded signal lines 63. The transmitting and receiving circuit 61 is provided with a transmission channel 65 and a reception channel 66 in each of the selecting switches 64. The transmission channels 65 and the reception channels 66 are connected in parallel in the selecting switches 64. The selecting switches 64 alternatively connect the transmission channels 65 or the reception channels 66 to the multiplexer 59. Pulsers 69 are provided in the transmission channels 65. The pulsers 67 output a pulse signal at a frequency corresponding to the resonance frequency of the vibrating films 52. Amplifiers 68, low-pass filters (LPF) 69, and analog-digital converters (ADC) 71 are incorporated in the reception channels 66. A detection signal of each of the elements 23 is amplified, and converted into a digital signal.

The transmitting and receiving circuit 61 is provided with a driving/receiving circuit 72. The transmission channels 65 and the reception channels 66 are connected to the driving/receiving circuit 72. The driving/receiving circuit 72 controls the pulsers 67 simultaneously depending on the state of scanning. The driving/receiving circuit 72 receives a digital signal of an output signal depending on the state of scanning. The driving/receiving circuit 72 is connected to the multiplexer 59 through a control line 73. The multiplexer 59 conducts an interconnection control based on a control signal supplied from the driving/receiving circuit 72.

A processing circuit 74 is incorporated in the device terminal 12. The processing circuit 74 can be provided with, for example, a central processing unit (CPU) 74 and a memory. The entire operations of the ultrasonic diagnostic device 11 are controlled in accordance with processing of the processing circuit 74. The processing circuit 74 controls the driving/receiving circuit 72 in accordance with instructions inputted by a user. The processing circuit 74 generates an image in accordance with an output signal of the elements 23. The image is specified by drawing data.

A drawing circuit 75 is incorporated in the device terminal 12. The drawing circuit 75 is connected to the processing circuit 74. The display panel 15 is connected to the drawing circuit 75. The drawing circuit 75 generates a drive signal in accordance with drawing data generated in the processing circuit 74. The drive signal is sent to the display panel 15. As a result, an image is displayed on the display panel 15.

### (3) Operations of Ultrasonic Diagnostic Device

Next, the operations of the ultrasonic diagnostic device 11 will be briefly described. The processing circuit 74 switches an ultrasonic diagnostic mode and a sensitivity detection mode. In the sensitivity detection mode, a deterioration of the sensitivity of the piezoelectric element section 24 can be determined. When the processing circuit 74 selects the ultrasonic diagnostic mode, the processing circuit 74 provides an instruction to the driving/receiving circuit 72 for transmitting and receiving an ultrasonic wave. The driving/receiving circuit 72 supplies a control signal to the multiplexer 59 and supplies a drive signal to each of the pulsers 67. A pulse signal is outputted in response to the supply of the drive signal. The multiplexer 59 connects the ports of the group of ports 59a to the ports of the group of ports 59b in accordance with the instruction of the control signal. A pulse signal is supplied to elements 23 in each column through the upper electrode terminals 33, 35 and the lower electrode terminals 34, 36 in response to the selection of the ports. The vibrating film 53 vibrates in response to the supply of the pulse signal. As a result, an intended ultrasonic beam is generated toward an object (e.g., inside of human body).

After ultrasonic wave was transmitted, the selecting switches 64 are switched. The multiplexer 59 maintains the connection relation of the ports. The selecting switches 64 establish a connection between the reception channels 66 and the signal lines 63 instead of a connection between the transmission channels 65 and the signal lines 63. Reflected waves of the ultrasonic waves vibrate the vibrating film 43. As a result, an output signal is output from the elements 23. The output signal is converted into a digital signal, and sent into the driving/receiving circuit 72.

Transmission and reception of ultrasonic waves are repeated. For repeating transmission and reception of ultrasonic waves, the multiplexer 59 changes the connection relation of the ports. As a result, the line scanning or the sector scanning is realized. When scanning is completed, the processing circuit 74 generates an image based on the digital signal of the output signal. The generated image is displayed on the screen of the display panel 15.

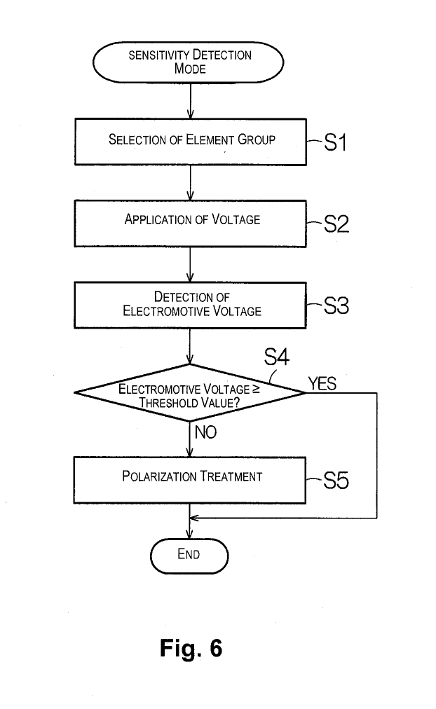

As shown in FIG. 6, the processing circuit 74 selects the sensitivity detection mode, the processing circuit 74 selects a particular one line of the elements 23 (hereinafter referred to as “target detection element line”) in Step S1. Here, as shown in FIG. 3, a group of the elements 23 connected to one of the second electric conductors 31 other than two of the second electric conductors 31 located in the outermost position can be selected as the target detection element line 76. The driving/receiving circuit 72 supplies a control signal to the multiplexer 59 in response to this selection. In the control signal, the target detection element line 76 and two lines of groups of elements 23 that are adjacent in both sides of the target detection element line 76 (hereafter referred to as “target drive element line”) are identified. The multiplexer 59 connects the ports of the group of ports 59a, which respectively connect to the target detection element line 76 and the target drive element lines 77, to any ports of the group of ports 59b in accordance with the instructions of the control signal. The target detection element line 76 is connected to the reception channels 66 and the two target drive element lines 77 are connected to the transmission channels 65 in response to the selection of the selecting switches 64.

The processing circuit 74 provides an instruction for a supply of a drive signal to the target drive element lines 77 in Step S2. The processing circuit 74 supplies a drive signal to two pulsers 67. The pulsers 67 output pulse signals (drive signals) in response to the supply of the drive signal. The pulse signals are supplied to the target drive element lines 77 through the first electric conductor 28 and the second electric conductor 31. Because of this, a voltage is applied to the respective piezoelectric element sections 24 in the target drive element lines 77. At this time, the driving/receiving circuit 72 functions as an input section to input a drive signal to the piezoelectric element sections 24 of a part of the elements 23.

The piezoelectric films 27 are deformed in response to the supply of the drive signal in the target drive element lines 77. The deformation of the piezoelectric films 27 leads to vibration, that is, the deformation of the vibrating films 51. As shown in FIG. 7, the partition walls 47 of the substrate 44 vibrate in response to the vibration of the vibrating films 51 of the target drive element lines 77. The stresses are generated in the piezoelectric films 27 of the target detection element line 76 in response to this vibration. An electromotive voltage is generated in the piezoelectric films 27 in response to the generation of the stresses. The electromotive voltage is outputted as an output signal. The target detection element line 76 is adjacent to the target drive element lines 77 so that the vibrating films 51 of the target drive element lines 77 securely leads to the deformation of the vibrating films 51 of the target detection element line 76.

The processing circuit 74 provides an instruction to the target detection element line 76 to receive an output signal in Step S3. In this time, the driving/receiving circuit 72 functions as a detection section to detect the vibration of the piezoelectric element section 24 of the target detection element line 76, that is, the piezoelectric elements, in which the drive signal is not inputted, among the piezoelectric elements while the drive signal is inputted to the piezoelectric element sections 24 of the target drive element lines 77, that is, a part of piezoelectric elements among the piezoelectric elements. The processing circuit 74 identifies a detection value of the output signal. The identified detection value is compared with a threshold value, which is preliminary determined in Step S4. The threshold value may be preliminary stored in a memory section as a memory of the processing circuit 74. When the detection value is more than the threshold value, it is determined as a good sensitivity. The sensitivity detection mode is end. When the detection value is less than the threshold value, the processing circuit 74 determines the low amount of polarization of the piezoelectric films 27. The deterioration of the sensitivity of the piezoelectric element section 24, that is, “abnormality” is determined. Because of this, when the “abnormality” is detected, the processing circuit 74 provides an instruction to execute a polarization process in Step S5. When the polarization process is executed, a voltage for polarization is supplied to the respective polarization body films 27. The polarization is realized in response to the application of the voltage for polarization in the piezoelectric films 27.

As described above, in the element unit 17, the polarization is established in the respective piezoelectric films 27 prior to use. The amount of polarization is reduced over time. As a result, the sensitivity of the elements 23 is deteriorated. Therefore, when voltage for polarization is supplied to the piezoelectric films 27 again in the case that the low sensitivity of the elements 23 is detected, a sufficient polarization can be established again in the piezoelectric films 27. The elements 23 can be recovered in the state of good sensitivity.

In the element unit 17, the elements 23 of the entire element array 22 execute the transmission and the reception of the ultrasonic waves for the ultrasonic diagnostic. The respective elements 23 switch between the transmission and the reception of the ultrasonic waves. The elements 23 emit an ultrasonic beam from the vibrating film 51 at the time of transmission. At the time of reception, the vibration of the vibrating film 51 is generated by the ultrasonic wave reflected from the object. The output signal is outputted from the elements 23 in response to the reflected ultrasonic wave. And, three lines of the group of elements 23 in the element array 22 are used for the judgment of the sensitivity. Therefore, a dedicated structure is not required to be added for the judgment of the sensitivity. The judgment of the sensitivity can be easily realized.

In this example, a deformation force is applied to the vibrating film 51 in the one line of the elements 23, which is the detection target for the judgment of the sensitivity, from the two lines of the elements 23 in both sides. Therefore, it can increase the stress of the piezoelectric films 27, which are the detection target, at one time of the supply of the drive signal compare to the case that a deformation force is simply applied from one line of the elements 23 in a single side. As a result, the accuracy of the judgment can be improved. In the element unit 17, the judgment of the sensitivity in each line of the all lines can be executed. In this case, in the lines in both sides of the element array 22, a deformation force is applied only from one line of the elements 23 in a single side to the piezoelectric films 27, which are the detection target. Also, the lines in the both sides of the element array 22 receive the drive voltage only for the judgment of the sensitivity, and it may not be used for the ultrasonic diagnostic mode.

Regarding the determination of the low amount of the polarization, in addition to the comparison between the detection value and the threshold value of Step S4, a change rate of a signal waveform of an output signal may be observed. For example, when a signal level of the start is more than the threshold value, it can be determined as a good sensitivity. When the signal level of the start is less than the threshold value, it can be determined as the low amount of the polarization. The signal level of the start can be identified based on the size of the signal level detected in a predetermined time interval.

Furthermore, instead of the polarization process of Step S5, the processing circuit 74 may generate a notification signal in response to the detection of the “abnormality”. For example, as a notification, an image signal displaying a deterioration of the sensitivity can be included. The image signal can be sent to the drawing circuit 75. The deterioration of the sensitivity can be notified to the user in the screen-display of the display panel 15. Because of this, the user can recognize the sensitivity deterioration of the piezoelectric film 27. In response to the notification, the probe head 13b or the element unit 17 may be replaced, or the polarization process of the piezoelectric film 27 can be executed through an external device.

In the notification signal, in addition to the above described image signal, an image signal indicating a size of an electromotive voltage may be included. The size of the electromotive voltage is shown in the screen-display of the display panel 15 to the user. The user can determine whether or not the amount of the polarization is an appropriate based on the size of the electromotive voltage. For an output of the notification signal, the processing circuit 74 can outputs an integral signal of a drive period that inputs a drive signal to the piezoelectric element section 24 of the target drive element lines 77. Because of this, an integral value of output signals for the drive period can be obtained.

### (4) Manufacturing Method of Ultrasonic Transducer Element Unit

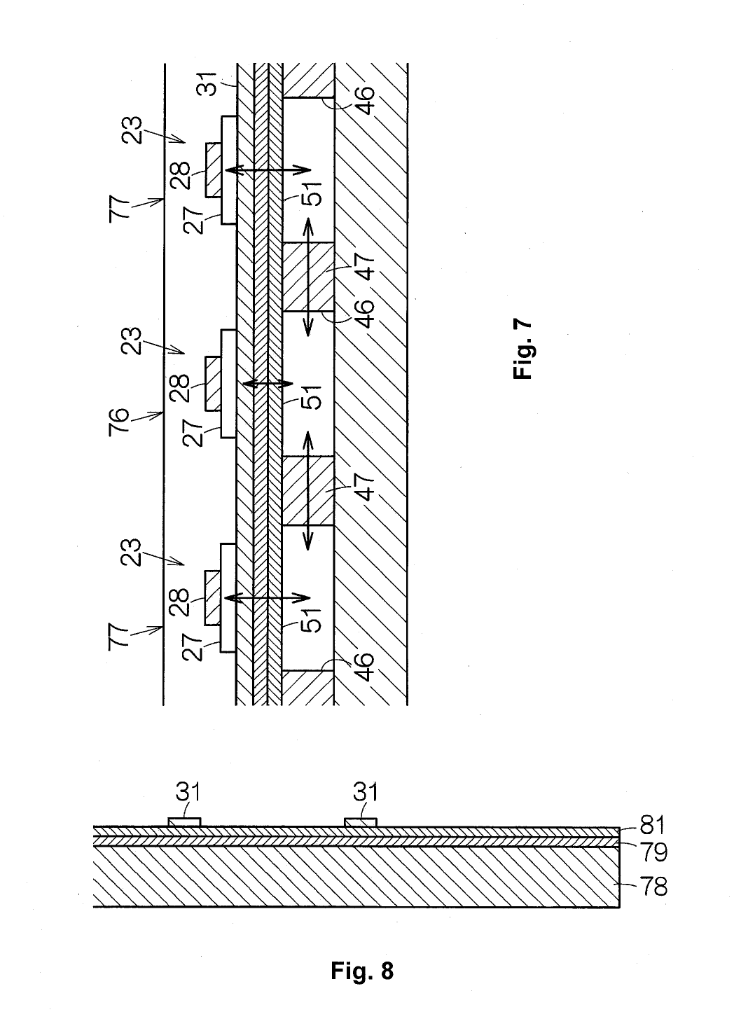

As shown in FIG. 8, the second electric conductors 31 and the lower electrode terminals 34, 36 (not shown in FIG. 8 or later drawings) are formed on the surface of the silicon wafer 78 in the respective element units 17. Prior to the formation of the second electric conductors 31 and the lower electrode terminals 34, 36, a silicon oxide film 79 and a zirconium oxide film 81 are consistently formed on the surface of the silicon wafer 78. A conducting film is formed on the surface of the zirconium oxide film 81. The conducting film is composed of the layered film of titanium, iridium, platinum, and titanium. The second electric conductors 31 and the lower electrode terminals 34, 36 are formed from the conducting film based on the photorefractive technology.

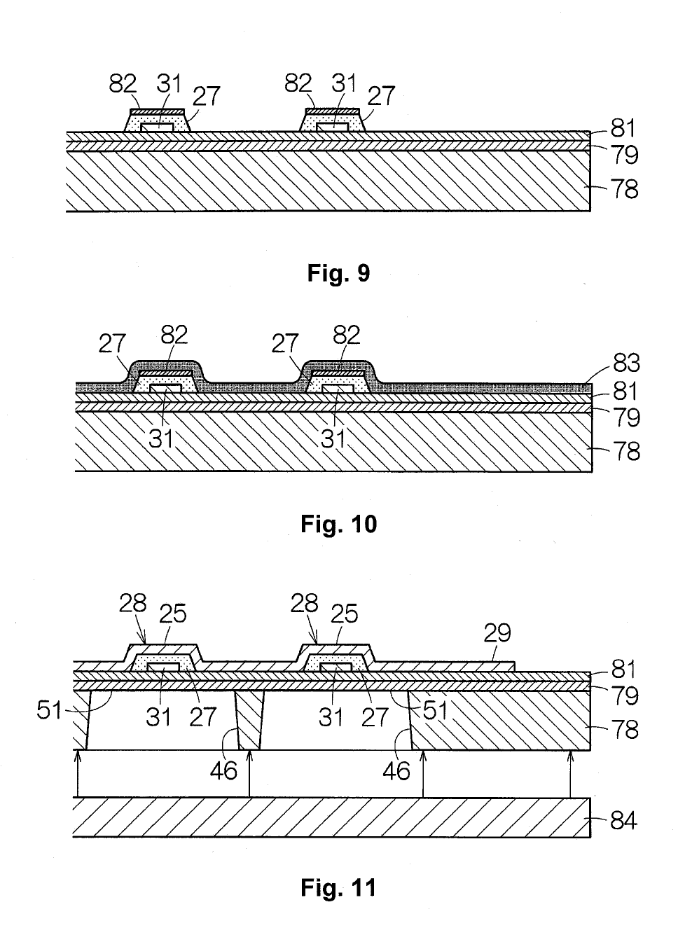

As shown in FIG. 9, the piezoelectric films 27 and the first conducting films 82 are formed on the surface of the second electric conductor 31 in the respective elements 23. A piezoelectric material film and a film of an electrical conducting material are deposited on the surface of the silicon wafer 78 to form the piezoelectric film 27 and the first conducting film 82. The piezoelectric material film is configured by the PZT film. The film of the electrical conducting material is configured by the iridium film. The piezoelectric film 27 and the first conducting film 82 are formed from the piezoelectric material film and the film of the electrical conducting material in the respective elements 23 based on the photorefractive technology.

Next, as shown in FIG. 10, an electric conducting material film 83 is formed on the surface of the silicon wafer 78. The electric conducting material film 83 covers the respective first conducting films 82. The adjacent first conducting films 82 are reciprocally connected by the film 83. And, the second conducting film is formed from the film 83 based on the photorefractive technology. The second conducting film extends in a direction intersecting to the first electric conductor 31, and crosses the first electric conductor 31 in sequence. The second conducting film is reciprocally connected to the first conducting film 82 in the direction of rows in the element array 22. The second conducting film forms the second electric conductor 31, the extraction wirings 29, and the upper electrode terminals 33, 35. A part of the second conducting film forms the upper electrodes 25 with the first conducting films 82 by overlapping with the first conducting film 82.

After that, as shown in FIG. 11, the openings 46 are formed in the array pattern from the back surface of the silicon wafer 78. An etching treatment is applied to form the openings 46. The silicon oxide film 79 functions as an etching-stop layer. The vibrating film 51 is partitioned into the silicon oxide film 79 and the zirconium oxide film 81. After the formation of the openings 46, the surface of a wafer for reinforcing plate 84 is overlapped with the back surface of the silicon wafer 78. Prior to the overlapping, the wafer 84 is maintained in the handling mechanism or on the stage. For example, a rigid insulating substrate can be used for the wafer 84. A silicon wafer can be used for the insulating substrate. For example, adhesive can be used for bonding. After the bonding, the respective element units 17 are cut out from the silicon wafer 78. A polarization treatment is applied in the element units 17 that were cut out. A voltage for polarization is applied to the respective piezoelectric films 27.

### (5) Ultrasonic Transducer Element Unit According to the Second Embodiment

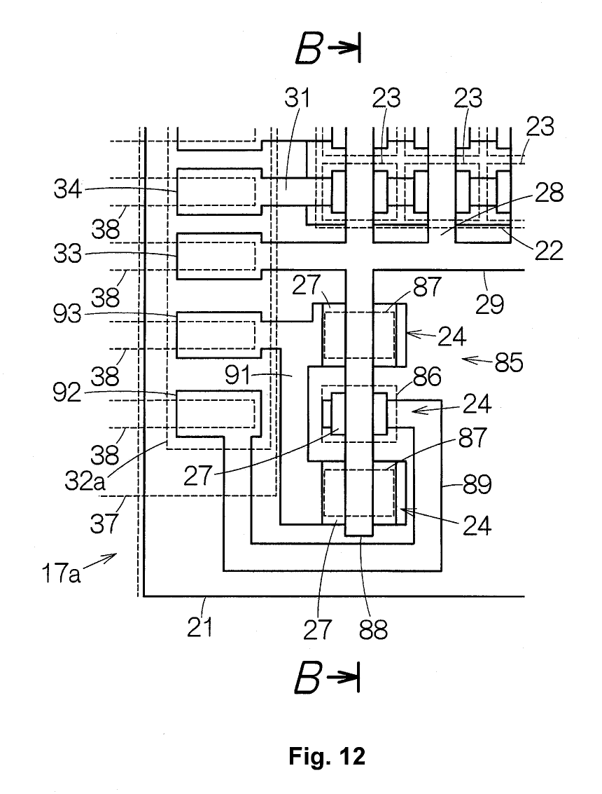

FIG. 12 schematically shows an ultrasonic transducer element unit 17a according to the second embodiment. In the second embodiment, in addition to the above described element array 22 on the substrate base 21, a piezoelectric element set 85 dedicated for the sensitivity detection mode is formed. As described above, the element array 22 is configured by the arrangement of the first elements 23. The piezoelectric element set 85 is arranged in the outside of the outline of the element array 22. The piezoelectric element set 85 is provided with one second element 86 and a pair of the third elements 87. The second element 86 functions as an element dedicated for the detection. The third elements 87 function as an element dedicated for the drive. The second element 86 is arranged between the two third elements 87. The second element 86 and the third elements 87 are provided with the piezoelectric element sections 24 in the same manner as the first elements 23. The piezoelectric element sections 24 are provided with the upper electrode 25, the piezoelectric film 27, and the lower electrode 26. The second element 86 is formed in the same structure as the first elements 23.

A first auxiliary electric conductor 88 is formed on the surface of the substrate base 21. The first auxiliary electric conductor 88 is mutually allocated to the second element 86 and the third elements 87. The first auxiliary electric conductor 88 is mutually connected to the second element 86 and the third elements 87 of the piezoelectric films 27. The first auxiliary electric conductor 88 forms the upper electrode 25 in the respective elements 86, 87. One end of the first auxiliary electric conductor 88 is connected to, for example, the extraction wiring 29. The first auxiliary electric conductor 88 may be formed in the same material as the first electric conductor 28 and the extraction wiring 29.

A second auxiliary electric conductor 89 is formed on the surface of the substrate. The second auxiliary electric conductor 89 is connected to the second element 86 and the piezoelectric films 27. The second auxiliary electric conductor 89 forms the second element 86 and the lower electrode 26. Because of this, in the second element 86, the drive voltage is applied to the piezoelectric films 27 from the first auxiliary electric conductor 88 and the second auxiliary electric conductor 89. The second auxiliary electric conductor 89 may be formed in the same material as the second electric conductors 31.

A third auxiliary electric conductor 91 is formed on the surface of the substrate base 21. The third auxiliary electric conductor 91 is mutually connected to the third elements 87 of the piezoelectric films 27. The third auxiliary electric conductor 91 forms the lower electrodes 26 of the third elements 87. Because of this, in the third elements 87, the drive voltage is applied to the piezoelectric films 27 from the first auxiliary electric conductor 88 and the third auxiliary electric conductor 91. The third auxiliary electric conductor 91 can be formed in the same material as the second electric conductors 31.

A first auxiliary electrode terminal 92 and a second auxiliary electrode terminal 93 are provided in the first terminal array 32a. The first auxiliary electrode terminal 92 is electrically connected to the second auxiliary electric conductor 89. The first auxiliary electrode terminal 92 and the second auxiliary electric conductor 89 may be unified. The second auxiliary electrode terminal 93 is electrically connected to the third auxiliary electric conductor 91. The second auxiliary electrode terminal 93 and the third auxiliary electric conductor 91 may be unified. The first auxiliary electrode terminal 92 and the second auxiliary electrode terminal 93 are respectively connected to the first wiring plate 37 and the first signal lines 38.

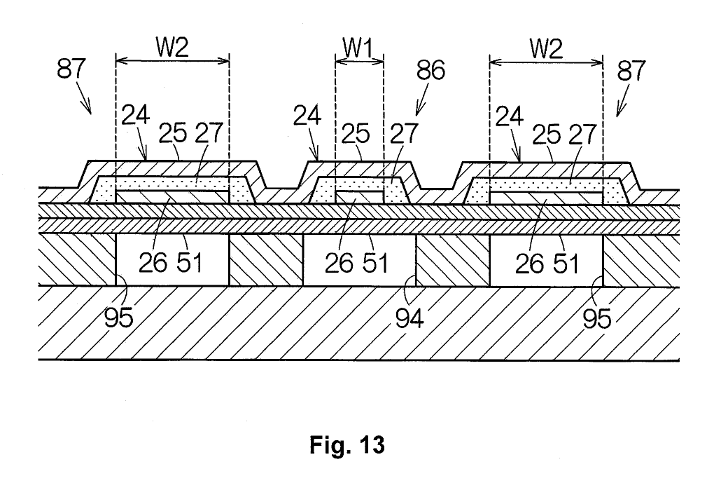

As shown in FIG. 13, in addition to the above described the first openings 46, a second opening 94 and third openings 95 are formed in the substrate 44. The second opening 94 partitions the vibrating film 51 of the second element 86. The third openings 95 partition the vibrating film 51 of the third element 87. Therefore, the second opening 94 and the third openings 95 are arranged in the outside of the outline of the element array 22. The third openings 95 are arranged closer to the second opening 94 than the first openings 46. The piezoelectric element section 24 of the third elements 87 is arranged on the second opening 94. The piezoelectric element section 24 of the third elements 87 is arranged on the third openings 95. Because of this, the piezoelectric element sections 24 of the second element 86 and the third elements 87 are respectively connected to the corresponding vibrating film 51.

At this point, the piezoelectric element sections 24 of the third elements 87 are formed in an area larger than the piezoelectric element sections 24 of the second elements 23, 86. Specifically, the piezoelectric element section 24 of the third elements 87 in the direction intersecting a central axis of the outline of the second element 86 has the second width W2 which is larger than the first width W1 of the piezoelectric element section 24 of the second element 86. In addition, the piezoelectric element sections 24 of the third elements 87 expand to the outside of the outline of the third openings 95. That is, the piezoelectric element sections 24 of the third elements 87 cross the third openings 95 and connect on the other side of the substrate 44. A size of the piezoelectric element section 24 is defined in an area wedged between the upper electrode 25 and the lower electrode 26 in the piezoelectric film 27.

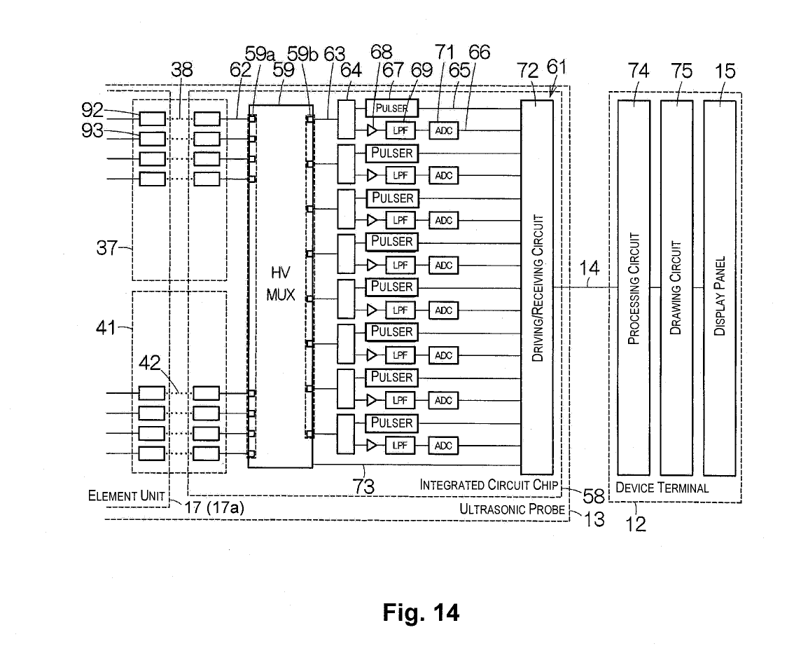

As shown in FIG. 14, in an integrated circuit chip 58 of the ultrasonic diagnostic device 11 that uses the element unit 17a according to the second embodiment, in addition to the above described upper electrode terminals 33, 35 and the lower electrode terminals 34, 36, the first auxiliary electrode terminals 92 and the second auxiliary electrode terminals 93 are connected to a group of ports 59a of a multiplexer 59 through wirings 62. When the sensitivity detection mode is selected in the processing circuit 74, the first auxiliary electrode terminals 92 are connected to the reception channels 66 and the second auxiliary electrode terminals 93 are connected to the transmission channels by the functions of the multiplexer 59. Other configurations and operations are the same as the description above.

When the processing circuit 74 selects the ultrasonic diagnostic mode, in the same manner as the above description, the drive signal is supplied to the first elements 23 in the element array 22 through the upper electrode terminals 33, 35 and the lower electrode terminals 34, 36. The line scanning or the sector scanning is realized by the function of the multiplexer 59. An image is displayed on the screen of the display panel 15 in response to the detection signal.

When the processing circuit 74 selects the sensitivity detection mode, the driving/receiving circuit 72 supplies a control signal to the multiplexer 59. In the control signal, the second element 86 and the third elements 87 are identified. The multiplexer 59 connects the group of ports 59a, which connects to the first auxiliary electrode terminals 92 and the second auxiliary electrode terminals 93, to any ports of the group of ports 59b in accordance with the instruction of the control signal. The first auxiliary electrode terminals 92 are connected to the reception channels 66, and the second auxiliary electrode terminals 93 are connected to the transmission channels 65 in response to the selection of the selecting switches 64.

The processing signal 74 instructs the third elements 87 to supply a drive signal. The processing circuit 74 supplies a drive signal to the pulsers 67. The pulsers 67 output a pulse signal (drive signal) in response to the supply of the drive signal. The pulse signal is supplied to the third elements 87 through the first auxiliary electric conductor 88 and the second auxiliary electric conductor 89.

The piezoelectric films 27 are deformed in response to the supply of the drive signal in the third elements 87. The deformation of the vibrating films 51 of the third elements 87 leads to the deformation in the vibrating film 51 of the second element 86. A stress is generated in the piezoelectric film 27 of the second element 86 in response to this deformation. An electromotive voltage is generated in the piezoelectric film 27 in response to the generation of the stress. The electromotive voltage is outputted as an output signal.

The processing circuit 74 instructs the second element 86 to receive an output signal. The output signal is converted to a digital signal and sent to the driving/receiving circuit 72. The processing circuit 74 identifies the detection value of the output signal. The identified detection value is compared with a threshold value that was preliminary set. When the detection value is more than the threshold value, the sensitivity detection mode is end. When the detection value is less than the threshold value, the processing circuit 74 determines the low amount of the polarization of the piezoelectric film 27. Generally, when the low sensitivity of the piezoelectric element section 24 in the second element 86, that is, “abnormality” is determined, in the same manner as the above description, the processing circuit 74 may instruct an execution of the polarization treatment or may generate a notification signal.

Here, the second element 86 has the same structure as the first elements 23. The second opening 94 has the same shape as the first openings. The vibrating films 51 have the same shape and the same film thickness. The piezoelectric element sections 24 have the same structure. In this way, the characteristics of the second element 86 can be easily related to the characteristics of the first elements 23. The characteristics of the second element 86 can be reflected from the characteristics of the first elements 23 with high accuracy. The variability of the characteristics in the respective elements in the element array 22 is small so that when the characteristics are identified in one second element 86, the characteristics of all of the first elements 23 can be presumed.

In the piezoelectric element set 85, the deformation force is applied from the both sides of the third elements 87 to the vibrating film 51 of the second element 86, which is a detection target. Therefore, the stress of the piezoelectric film 27 as a detection target can be increased by the supply of the drive signal at once compare to the case that the deformation force is simply applied from one side. The electromotive voltage of the piezoelectric film 27 is increased. As a result, the accuracy of the judgment can be improved. In addition, the vibrating films 51 of the third elements 87 can increase the deformation of the piezoelectric films 27. The accuracy of the judgment can be more improved. However, as long as the deformation force is sufficiently applied to the piezoelectric film 27 of the second element 86 from the piezoelectric of the third elements 87, the vibrating films 51 in the third elements 87 can be omitted.

In the piezoelectric element set 85, the piezoelectric element sections 24 of the third elements 87 are formed larger than the piezoelectric element section 24 of the second element 86. Thus, larger deformation force is applied to the second element 86. As a result, the accuracy of the judgment can be improved. The piezoelectric element set 85 is arranged in the outside of the outline of the element array 22 so that the expansion of the third elements 87 does not affect to the first elements 23 in the element array 22.

In addition, the piezoelectric element sections 24 of the third elements 87 expand to the outside of the outline of the third openings 95. The deformation of the third elements 87 can be directly transmitted to the substrate 44 of the surrounding of the second opening 94, that is, the partition wall 47. Therefore, the deformation of the third elements 87 can be efficiently transmitted to the second element 86 compare to the case that the deformation of the third elements 87 is transmitted to the substrate 44 of the surrounding of the second opening 94 through the vibrating film 51. As a result, the stress of the second element 86 can be increased. The accuracy of the judgment can be improved.

### (6) Ultrasonic transducer Element Unit According to the Third Embodiment

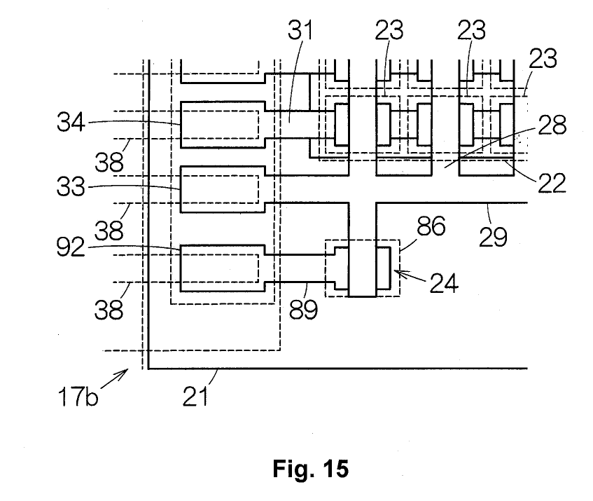

FIG. 15 schematically shows a structure of an element unit 17b according to the third embodiment. In the third embodiment, in addition to the above described piezoelectric element set 85 on the substrate base 21, a single second element 86 dedicated for the sensitivity detection mode is formed on the substrate base 21. The second element 86 is arranged the outside of the outline of the element array 22. The second element 86 is provided with the first elements 23, as well as, the piezoelectric element section 24. The second element 86 is formed in the same structure as the first elements 23. The second auxiliary electric conductor 89 is connected to the piezoelectric film 27 of the second element 86. The first auxiliary electrode terminal 92 is electrically connected to the second auxiliary electric conductor 89. Also, the structure and the operations are the same as the description above.

When the processing circuit 74 selects the sensitivity detection mode, the driving/receiving circuit 72 supplies a control signal to the multiplexer 59. In the control signal, the second element 86 and one line of the group of the first elements 23, which is the closest to the second element 86, are identified (hereinafter referred to as “target drive element line”). The multiplexer 59 respectively connects the ports of the group of ports 59a, which connects to the target drive element line and the second auxiliary electrode terminal 93, to any ports of the group f ports 59b in accordance with the instruction of the control signal. The first auxiliary electrode terminals 92 are connected to the reception channels 66 and the second auxiliary electrode terminals 93 are connected to the transmission channels 65 in response to the selection of the selecting switches 64.

### (7) Ultrasonic Diagnostic Device According to Another Embodiment

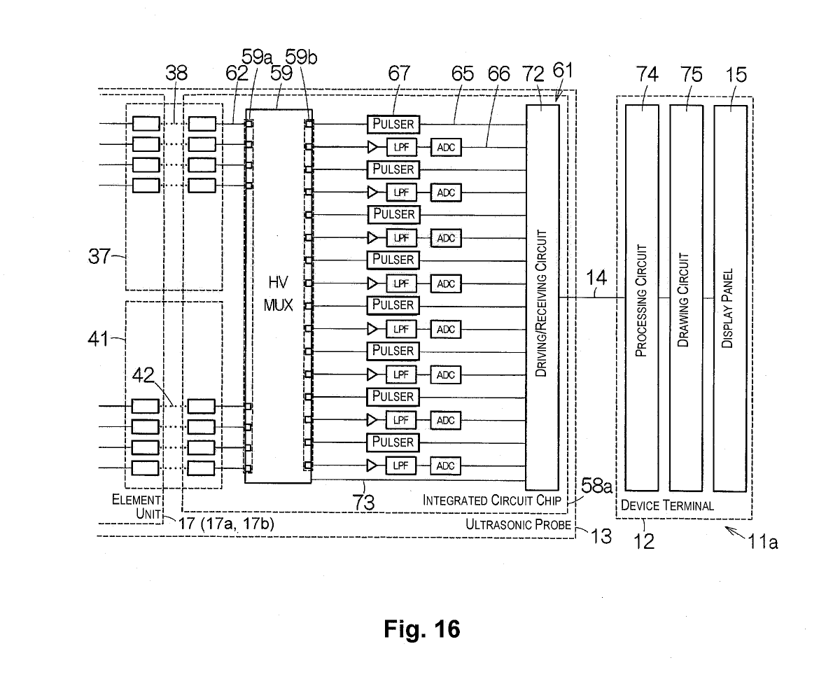

FIG. 16 schematically shows a circuit configuration of an ultrasonic diagnostic device 11a according to another embodiment. In the ultrasonic diagnostic device 11a, an integrated circuit chip 58a is connected to the element units 17, 17a, 17b. In the element units 17, 17a, 17b, the elements for transmission 23 and the elements for reception 23 are allocated in each line of the element array 22. For example, the lines for transmission and the lines for reception can be arranged alternatively. In the integrated circuit chip 58a, the transmission channels 65 and the reception channels 66 are respectively connected to the multiplexer 59. The multiplexer 59 connects the transmission channels 65 to the elements 23 in each line for transmission at the time of transmitting an ultrasonic wave. The multiplexer 59 connects the reception channels 66 to the elements 23 in each line for reception at the time of receiving the ultrasonic wave. The elements 23 connected to the transmission channels 65 are assigned for the transmission of the ultrasonic wave. The elements 23 connected to the reception channels 66 are assigned for the reception of the ultrasonic wave. Because of this, the transmission and the reception of the ultrasonic wave are assigned in each of the respective elements 23 so that the respective elements 23 can be adjusted to be specialized for the transmission or the reception of the ultrasonic wave. As a result, the reception sensitivity of the ultrasonic wave can be improved.

By the way, the present embodiments were described above in detail, but it will be apparent to those skilled in the art that various modifications can be made in a scope not substantially deviating from the subject matter and the effect of the present invention. Therefore, such changes and modifications are included in the scope of the invention. For example, the terms used in the specification or the drawings at least once together with a different term having a broader or similar meaning can be replaced with the different term in any portion of the specification or the drawings. Also, the configurations and the operations of the ultrasonic diagnostic device 11, 11a, the ultrasonic probe 13, the prove head 13b, the element units 17, 17a, the elements 23, 86, 87, the integrated circuit 58, 58a, and the like are not limited to the present embodiment, and various modifications are possible.

### General Interpretation of Terms

In understanding the scope of the present invention, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. Also, the terms “part,” “section,” “portion,” “member” or “element” when used in the singular can have the dual meaning of a single part or a plurality of parts. Finally, terms of degree such as “substantially”, “about” and “approximately” as used herein mean a reasonable amount of deviation of the modified term such that the end result is not significantly changed. For example, these terms can be construed as including a deviation of at least ±5% of the modified term if this deviation would not negate the meaning of the word it modifies.

While only selected embodiments have been chosen to illustrate the present invention, it will be apparent to those skilled in the art from this disclosure that various changes and modifications can be made herein without departing from the scope of the invention as defined in the appended claims. Furthermore, the foregoing descriptions of the embodiments according to the present invention are provided for illustration only, and not for the purpose of limiting the invention as defined by the appended claims and their equivalents.

What is claimed is:

1. An ultrasonic transducer device comprising:
a substrate having a plurality of openings;
a vibrating film provided in each of the openings to cover a corresponding one of the openings;
a piezoelectric element provided in each of the openings on the vibrating film;
an input section configured and arranged to input a drive signal to a part of piezoelectric elements among the piezoelectric elements; and
a detection section configured and arranged to detect vibration of the piezoelectric elements, in which the drive signal is not inputted, among the piezoelectric elements while the drive signal is inputted to the part of the piezoelectric elements among the piezoelectric elements.

2. The ultrasonic transducer device according to claim 1, further comprising
a control processing section configured to determine sensitivity of the piezoelectric elements based on a detection result that vibration of the piezoelectric element in which the drive signal is not inputted was detected.

3. The ultrasonic transducer device according to claim 1, wherein
the plurality of openings is arranged in a matrix pattern or a line pattern in plan view along a thickness direction of the substrate, and
the detection section is configured and arranged to detect vibration of a piezoelectric element that is located adjacent to the part of piezoelectric elements, in which the drive signal is inputted, among the piezoelectric elements, in which the drive signal is not inputted.

4. The ultrasonic transducer device according to claim 3, wherein
the detection section is configured and arranged to detect vibration of a piezoelectric element that is located between two piezoelectric elements, in which the drive signal is inputted, among the piezoelectric elements, in which the drive signal is not inputted, and is also located adjacent to the two piezoelectric elements.

5. The ultrasonic transducer device according to claim 2, wherein
when the control processing section determines that a sensitivity of a piezoelectric element, in which the drive signal is not inputted, is less than a predetermined value, the control processing section is configured to supply a voltage for polarization to the piezoelectric element, in which the drive signal is not inputted.

6. The ultrasonic transducer device according to claim 2, wherein
when the control processing section determines that a sensitivity of a piezoelectric element, in which the drive signal is not inputted, is less than a predetermined value, the control processing section is configured to output a notification signal indicating that the sensitivity is less than the predetermined value.

7. The ultrasonic transducer device according to claim 1, further comprising
a control processing section configured to output a notification signal based on a detection result that vibration of a piezoelectric element, in which the drive signal is not inputted, was detected.

8. An ultrasonic transducer device comprising:
a substrate having a first opening, a second opening, and a partition part, which is wedged between the first opening and the second opening;
a vibrating film provided in each of the first opening and the second opening to cover a corresponding one of the first opening and the second opening;
a first piezoelectric element provided on the vibrating film including a position overlapped with the first opening in plan view as viewed along a thickness direction of the substrate;
a second piezoelectric element provided on the vibrating film including a position overlapped with the second opening in plan view as viewed along the thickness direction of the substrate;
an input section configured and arranged to input a drive signal to the first piezoelectric element; and
a detection section configured and arranged to detect vibration of the second piezoelectric element, in which the drive signal is not inputted, while the drive signal is inputted to the first piezoelectric element;
wherein the partition part has a shape that a thickness in the thickness direction of the substrate is larger than a minimum value of distance between the first opening and the second opening in plan view as viewed along the thickness direction of the substrate.

9. An ultrasonic transducer device comprising:
a substrate having a plurality of first openings arranged in a matrix pattern or a line pattern, and a second opening arranged in an outside of an outline of an area where the first openings are arranged;
a vibrating film provided in each of the first openings and the second opening to cover a corresponding one of the first openings and the second opening;
a first piezoelectric element provided on the vibrating film in each of the first openings in plan view as viewed along a thickness direction of the substrate;
a second piezoelectric element provided on the vibrating film including a position overlapped with the second opening in plan view as viewed along the thickness direction of the substrate;
an input section configured and arranged to input a drive signal to the first piezoelectric elements; and
a detection section configured and arranged to detect vibration of the second piezoelectric element, in which the drive signal is not inputted, while the drive signal is inputted to the first piezoelectric elements.

10. The ultrasonic transducer device according to claim 9, wherein
the first openings and the second opening are formed in the same shape, and
the first piezoelectric element and the second piezoelectric element are formed in the same structure.

11. An ultrasonic transducer device comprising:
a substrate having
a plurality of first openings arranged in a matrix pattern or a line pattern,
a second opening arranged in an outside of an outline of a region where the first openings are arranged, and
a third opening arranged in the outside of the outline of the region where the first openings are arranged in a position closer to the second opening than to the first openings;
a vibrating film provided in each of the first openings, the second opening, and the third opening to cover a corresponding one of the first openings, the second opening, and the third opening;
a first piezoelectric element provided on the vibrating film in each of the first openings in plan view of a thickness direction of the substrate;
a second piezoelectric element provided on the vibrating film including a position overlapped with the second opening in plan view as viewed along the thickness direction of the substrate;
a third piezoelectric element being provided on the vibrating film including a position overlapped with the third opening in plan view as viewed along the thickness direction of the substrate;
an input section configured and arranged to input a drive signal to the third piezoelectric element; and
a detection section configured and arranged to detect vibration of the second piezoelectric element, in which the drive signal is not inputted, while the drive signal is inputted to the third piezoelectric element.

12. The ultrasonic transducer device according to claim 11, wherein
the third piezoelectric element has a larger area than the second piezoelectric element in plan view as viewed along the thickness direction of the substrate.

13. The ultrasonic transducer device according to claim 9, further comprising
a control processing section configured and arranged to determine a sensitivity of the first piezoelectric element based on a detection result that vibration of the second piezoelectric element in which the drive signal is not inputted was detected.

14. The ultrasonic transducer device according to claim 13, wherein
when the control processing section determines that the sensitivity of the first piezoelectric element is less than a predetermined value, the control processing section is configured to supply a voltage for polarization to the first piezoelectric element.

15. The ultrasonic transducer device according to claim 13, wherein
when the control processing section determines that the sensitivity of the first piezoelectric element is less than a predetermined value, the control processing section is configured to output a notification signal indicating that the sensitivity is less than the predetermined value.

16. A probe comprising:
the ultrasonic transducer device according to claim 1; and
a case supporting the ultrasonic transducer device.

17. An electronic instrument comprising:
the ultrasonic transducer device according to claim 1; and
a processing part connected to the ultrasonic transducer device to process an output of the ultrasonic transducer device.

18. An ultrasonic diagnostic device comprising:
the ultrasonic transducer device according to claim 1;
a processing part connected to the ultrasonic transducer device to process an output of the ultrasonic transducer device and to generate an image; and
a display device configured and arranged to display the image.

19. A probe head comprising:
the ultrasonic transducer device according to claim 1, and
a case supporting the ultrasonic transducer device.

20. An ultrasonic transducer device comprising:
a substrate having a plurality of openings partitioned by a partition part;
a vibrating film provided in each of the openings to cover a corresponding one of the openings;
a piezoelectric element provided in each of the openings on the vibrating film;
an input section configured and arranged to input a drive signal to a part of piezoelectric elements among a plurality of piezoelectric elements; and
a detection section configured and arranged to detect vibration of the piezoelectric element, in which the drive signal is not inputted, while the drive signal is inputted in the part of piezoelectric elements;
wherein the vibration of the vibrating film vibrated by inputting the drive signal to the part of piezoelectric elements deforms the partition part so as to vibrate the piezoelectric elements, in which the drive signal is not inputted.
